(19) United States
(12) Patent Application Publication
(10) Pub. No.: US 2002/0045678 A1
(43) Pub. Date: Apr. 18, 2002

(54) Dental restorative compositions and method of use thereof

(76) Inventors: Larry A. Lopez, Boerne, TX (US); Weitao Jia, Wallingford, CT (US); Shuhua Jin, Wallingford, CT (US)

Correspondence Address: CANTOR COLBURN, LLP, 55 GRIFFIN ROAD SOUTH, BLOOMFIELD, CT, 06002

(21) Appl. No.: 09/935,048

(22) Filed: Aug. 22, 2001

Related U.S. Application Data
(60) Provisional application No. 60/227,111, filed on Aug. 22, 2000.

(51) Int. Cl. A61K 6/08; C08K 3/00

(52) U.S. Cl. 523/116; 524/5

(57) ABSTRACT

A curable dental restorative composition having a curable, ethylenically unsaturated component, preferably a hydrophilic, methacrylate-containing monomer or oligomer component, a calcium silicate cement component, and a non-water curing component. Water and/or fillers may optionally be present. The use of curable monomers or oligomers as part of the hardening agent for the cement component results in improved setting time and physical properties in the cured product. Water and/or fillers may optionally be present.

### CROSS-REFERENCE TO RELATED APPLICATIONS

This application claims priority to U.S. application Ser. No. 60/227,111, filed Aug. 22, 2000, and U.S. Ser. No. 60/232,502 filed Sep. 13, 2000, both of which are incorporated by reference herein in their entirety.

### BACKGROUND OF THE INVENTION

1. Field of the Invention

This invention relates to compositions for restorative dentistry, and more particularly to curable compositions usable as restoring materials suitable for dental fillings, crowns and bridges, and especially for root canal sealing and pulp capping. These compositions have improved physical and mechanical properties, as well as improved setting time.

2. Brief Description of the Related Art

In recent years, materials used for dental restorations have comprised principally cured acrylate or methacrylate monomers or oligomers. Typical cured acrylate resinous materials are disclosed in U.S. Pat. No. 3,066,112 to Bowen, U.S. Pat. No. 3,179,623 to Bowen, U.S. Pat. No. 3,194,784 to Bowen, U.S. Pat. No. 3,751,399 to Lee et al. and U.S. Pat. No. 3,926,906 to Lee et al. An especially important curable methacrylate monomer is the condensation product of bisphenol A and glycidyl methacrylate, 2,240 -bis[4-(3-methacryloxy-2-hydroxy propoxy)-phenyl]-propane (hereinafter abbreviated “Bis-GMA”). Because acrylic resin systems alone are less than satisfactory, composite acrylic dental restorative materials containing resins and fillers were developed. The fillers are generally inorganic materials based on silica, silicate based glasses, or quartz.

Another type of dental restorative material are the glass ionomers, typically referred to as glass ionomer cements, wherein a poly(carboxylic acid) (such as a homo- or co-polymer of acrylic acid) is reacted with a fluoride ion leachable species (such as a fluoroaluminosilicate glass) in the presence of water to yield a crosslinked network structure. Because of the incorporation of the fluoride ion leachable species, glass ionomers are capable of providing long-term fluoride release.

The use of cementitious materials for dental restorations is also known. Portland cements in particular are disclosed to have use as filling and sealing materials for tooth cavities in U.S. Pat. Nos. 5,415,547 and 5,769,638. There are a number of drawbacks to cementitious materials, however, including slow setting times (on the order of 24 hours), and brittleness in the final product. Thus, while current cement-based materials may be of some utility as sealing materials, there accordingly remains a need in the art for cementitious materials with improved physical properties.

### SUMMARY OF THE INVENTION

The drawbacks and deficiencies of the prior art are remedied by a curable dental composition comprising a polymerizable, ethylenically unsaturated component, a calcium silicate cement component, and at least one non-water curing component. The curable dental composition can be self-curing and/or light curing, in conjunction with water curing. Such curable dental compositions are useful for a variety of dental treatments and restorative functions including crown and bridge materials (including temporary crown and bridge materials), sealants, fixing cements, inlays, onlays, veneer shells, and filling materials.

### DETAILED DESCRIPTION OF THE PREFERRED EMBODIMENTS

A curable dental restorative composition comprises a curable, ethylenically unsaturated component, preferably a hydrophilic, methacrylate-containing monomer or oligomer component, a calcium silicate cement component, and a non-water curing component. The use of curable monomers or oligomers as part of the hardening agent for the cement component results in improved setting time and physical properties in the cured product. In a further embodiment, the composition also comprises one or more fillers, for example barium glasses, calcium phosphates, and other fillers. These fillers promote the linkage between the polymerized component and the cement and therefore improve the mechanical strength of the composition.

The curable, ethylenically unsaturated component is preferably a monomer or oligomer containing at least two acrylate or methacrylate groups, and generally comprises viscous acrylate or methacrylate monomers such as those disclosed in U.S. Pat. No. 3,066,112 to Bowen, U.S. Pat. No. 3,179,623 to Bowen, U.S. Pat. No. 3,194,784 to Bowen, U.S. Pat. No. 3,751,399 to Lee et al., U.S. Pat. No. 3,926,906 to Lee et al., and commonly assigned U.S. Pat. Nos. 5,276,068 and 5,444,104 to Waknine, all of which are incorporated herein by reference. Other resin materials include, but are not limited to, urethane dimethacrylate (UDMA), diurethane dimethacrylate (DUDMA), polyurethane dimethacrylate (PUDMA), polyethylene glycol dimethacrylate (PEGDMA), and other monomers and oligomers known in the art. A particularly useful oligomer is disclosed in U.S. Pat. Nos. 5,276,068 and 5,444,104 to Waknine, being a polycarbonate dimethacrylate (PCDMA) which is the condensation product of two parts of a hydroxyalkylmethacrylate and one part of a bis(chloroformate).

Alternatively, or in addition, the polymerizable component may comprise one of the so-called “diluent” acrylate or methacrylate monomers. Suitable diluent monomers include those known in the art such as hydroxy alkyl methacrylates, for example 2-hydroxyethyl methacrylate and 2-hydroxypropyl methacrylate; ethylene glycol methacrylates, including ethylene glycol methacrylate, diethylene glycol methacrylate, tri(ethylene glycol) dimethacrylate and tetra(ethylene glycol) dimethacrylate; and diol dimethacrylates such as butanedimethacrylate, dodecanedimethacryalte, or 1,6-hexanedioldimethacrylate. Tri(ethylene glycol) dimethacrylate (TEGDMA) is particularly preferred.

Preferably, the polymerizable component is a hydrophilic, ethylenically unsaturated monomer, or comprises such a monomer. Suitable hydrophilic, ethylenically unsaturated monomers may have carboxyl, phosphoryl, sulfonyl, and/or hydroxyl functional groups, together with at least one ethylidenyl, acrylate, or methacrylate group. Examples of such hydrophilic monomers having at least one carboxyl group include but are not limited to methacrylic acid, maleic acid, p-vinylbenzoic acid, 11-methacryloyloxy-1,1-undecanedicarboxylic acid, 1,4-dimethacryloyloxyethylpyromellitic acid, 6-methacryloyloxyethylnaphthalene-1,2,6-tricarboxylic acid, 4-methacryloyloxymethyltrimellitic acid and the anhydride thereof, 4-methacryloyloxyethyltrimellitic acid (“4-MET”) and an anhydride thereof (“4-META”), 4-(2-hydroxy-3-methacryloyloxy)butyltrimellitic acid and an anhydride thereof, 2,3-bis(3,4-dicarboxybenzoyloxy)propyl methacrylate, methacryloyloxytyrosine, N-methacryloyloxyphenylalanine, methacryloyl-p-aminobenzoic acid, an adduct of 2-hydroxyethyl methacrylate with pyromellitic dianhydride (PMDM), and an adduct of 2-hydroxyethyl methacrylate with 3,3′,4,4′-benzophenonetetracarboxylic dianhydride (BTDA) or 3,3′,4,4′-biphenyltetracarboxylic dianhydride.

Presently preferred hydrophilic monomers include BPDM, the reaction product of an aromatic dianhydride with an excess of 2-HEMA (2-hydroxyethyl methacrylate), as disclosed in U.S. Pat. No. 5,348,988, which is incorporated by reference herein. Other presently preferred hydrophilic monomers include EDMT, the reaction product of 2-hydroxyethyl methacrylate (“2-HEMA”) with ethylene glycol bistrimellitate dianhydride; DSDM, the reaction product of 3,3′,4,4′-diphenylsulfone tetracarboxylic dianhydride and 2-HEMA; PMDM; and PMGDM, the adduct of pyromellitic dianhydride with glycerol dimethacrylate.

Another type of preferred hydrophilic, ethylenically unsaturated species includes the degradable macromonomers having terminal acrylate or methacrylate groups disclosed in DENTAL COMPOSITIONS COMPRISING DEGRADABLE POLYMERS AND METHODS OF MANUFACTURE THEREOF, filed Aug. 10, 2000, and claiming priority to U.S. provisional application Ser. No. 60/148,887, filed Aug. 13, 1999, and incorporated by reference herein in its entirety. Degradable macromonomers are manufactured by the polymerization of cyclic lactide, glycolide, or caprolactone in the presence of a compound having at least one active hydrogen and at least one acrylate or methacrylate functionality. Preferred active hydrogen containing acrylate or methacrylate compounds comprise 2-hydroxyethyl methacrylate, hydroxypolyethyl methacrylate, phenoxy-2-hydroxypropyl methacrylate, and the like. Preferred co-polymerizable acrylate or methacrylate monomers include diluent monomers such as 1,6-hexanediol dimethacrylate, triethylene glycol trimethacrylate, and 2-hydroxyethyl methacrylate. Degradable macromonomers can also be manufactured by the esterification of hydroxyl-group(s) terminated macromonomers of the above-mentioned hydroxy acids with acrylic acid, methacrylic acid, and their derivatives.

The polymerizable component is used in amounts of about 5 to about 90 weight percent, preferably about 20 to about 70, and more preferably about 30 to about 60 weight percent based on the total weight of the polymerizable component, cement component, and curing component.

As used herein, the “cement component” is a dry cement settable in an aqueous environment. The preferred cement component is a calcium silicate cement commonly known as Portland cement. The process of making Portland cement is well known, and it can be purchased from any number of manufacturers under various trade names. The basic raw materials for Portland cement are lime (CaO), silica (SiO₂), alumina (Al₂O₃), and iron oxide (Fe₂O₃), appropriately proportioned to produce various types of Portland cement. In the manufacture of Portland cement, the selected raw materials are crushed, ground, and then blended in the desired proportions. The mixture is then fed into a rotary kiln where it is heated to temperatures of up to 1400° to 1650° C., cooled, and subsequently pulverized. A small amount of gypsum (CaSO₄.2H₂O) may be added to the cement to control the setting time. The resulting cement consists principally of tricalcium silicate (3CaO.SiO₂), dicalcium silicate (2CaO.SiO₂), tricalcium aluminate (3CaO.Al₂O₃), and tetracalcium aluminoferrite (4CaO.Al₂O₃.Fe₂O₃). It is common, however, to report Portland cement compositions on the basis of the corresponding oxides.

The principal component of Portland cement by weight is calcium, which is present in amount of about 50 to about 75 weight percent, preferably about 65 weight percent of the total cement composition, calculated as calcium oxide (CaO). Silicon is present in an amount of about 15 to about 25 weight percent, preferably about 21 weight percent of the total cement composition, calculated on the basis of silicon dioxide (SiO₂). The combination of calcium and silicon components is present in an amount of about 70 to about 95 weight percent, preferably about 86 weight percent of the total cement composition, based on the corresponding oxides.

The suitability of a particular cement component for a given purpose is typically determined by a combination of its chemical component and its physical attributes, i.e. the manner and degree to which the cement is ground (granulation) and the resulting particle size. The fineness of a cement is indicated by the Blaine number, which represents the ratio of the particle surface area to its weight (square centimeters of surface per gram). Portland cements generally have a Blaine number in the range of 3,200 to 5,500 cm²/g or greater. Faster setting cements, like that preferably utilized in the present invention, have a Blaine number in the range of 4,000-5,500 cm²/g. The most preferable cement utilized in the present invention has a Blaine number in the range of 4,500-4,600 cm²/g.

In general, as defined for its typical use, there are five basic types of Portland cement. These are identified by the standard specifications promulgated by the American Society for Testing of Materials (ASTM). Type I is called normal Portland cement and is a general purpose cement suitable for all uses when the special properties of the other types are not required. Type I Portland cement is more generally available than are the other types of cement, and in its normal applications, is used where the heat generated by the hydration of the cement will not cause an objectionable rise in temperature. Such conditions are typical of the mouth, which would normally not necessitate the use of ASTM Types II through V. The preferred embodiment accordingly utilizes a Type I Portland cement having the following approximate composition by weight (calculated on the basis of the corresponding oxides): 21% SiO₂, 4% Al₂O₃, 5% Fe₂O₃, 65% CaO, 2% MgO, 2.5% SO₃, and 0.5% of alkalis such as Na₂O and/or K₂O. This Portland cement is commercially available as the Colton Fast-Set brand of the California Portland Cement Company.

Although the preferred embodiment thus comprises an ASTM Type I Portland cement, other types of hydraulic (water-settable) cements, particularly calcium silicate cements, may be suitable for the purposes described herein. In particular, Type III Portland cement is used when early strength is desired, which may be suitable for certain applications where early strength may be advantageous. Type IV is a low heat of hydration cement useful when the heat of hydration is critical. It would typically not be required in anatomical structures, but it may be useful, for example, if an additive were used that may be adversely affected by a higher heat of hydration.

The cement component is generally present in an amount from about 10 to about 95 percent by weight, preferably in an amount from about 20 to about 60 percent by weight, and even more preferably in an amount from about 30 to about 50 percent by weight of the polymerizable component, cement component, and curing component.

Depending on the particular application, various amounts of water (from 0 to 50% by weight of the dry cement component) may be utilized in the dental restorative composition. Enough water is added to the cement to give it a putty consistency, which then solidifies to a rock-like hardness. The water content is in the range of 10 to 40 weight percent, and most preferably three parts cement are used with one part water, or 25 weight percent of the dry cement component is water.

Use of water as a reactant in the hardening reaction offers significant advantages in dental restorative compositions. Calcium silicate cements harden upon reaction with water. The hardening reactions are complex, but principally involve the hydration of tricalcium silicate (3CaO.SiO₂) and dicalcium silicate (2CaO.SiO₂). The principal early reaction product is tricalcium silicate hydrate, a colloidal gel of extremely small particles (less than about one micron) that is most responsible for the early strength of Portland cement. The dicalcium silicate has a slower hydration reaction and is mainly responsible for strength increases beyond one week. Tricalcium aluminate, which plays a lesser role in the hardening process, hydrates rapidly also and contributes to early strength of development.

The curable dental restorative composition further includes a non-water curing component such as a light-curing and/or self-curing system. The light-cure system is selected from known light-activated polymerization initiators, including but not being limited to benzil, benzoin, benzoin methyl ether, DL-camphorquinone (CQ), and benzil diketones. Either UV-activated cure or visible light-activated cure (approx. 230 to 750 nm) is acceptable. The amount of photoinitiator is selected according to the curing rate desired. A minimally catalytically effective amount is generally about 0.01 percent by weight of the polymerizable components, and will lead to a slower cure. Faster rates of cure are achieved with amounts of catalyst in the range from greater than about 0.01 percent to about 5 percent by weight of the polymerizable component. It is furthermore preferred to employ an ultraviolet absorber in amounts ranging from about 0.01 to about 1.0 weight percent. Such UV absorbers are particularly desirable in the visible light curable components in order to avoid discoloration of the resin from any incident ultraviolet light. Suitable UV absorbers are the various benzophenones, particularly UV-5411 available from American Cyanamid Company. Light-cure systems are generally proved to the practitioner as one-part systems.

Alternatively, or in addition, the dental restorative composition may be formulated with a self-curing system. Self-curing components will generally contain free radical polymerization initiators such as, for example, a peroxide in amounts of about 0.01 to about 3.0 weight percent. Particularly suitable free radical initiators are lauryl peroxide, tributyl hydroperoxide and, more particularly benzoyl peroxide (BPO). Self-cure systems may further comprise an accelerator such as a tertiary amine, generally tertiary aromatic amines such as ethyl 4-(dimethylamino)benzoate (commonly known as AEDMAB”), 2-[4-(dimethylamino)phenyl]ethanol, N,N-dimethyl-p-toluidine (DMPT), bis(hydroxyethyl)-p-toluidine (DHEPT), and triethanolamine. Such accelerators are generally present in amounts from about 0.5 to about 4.0 percent by weight of the polymeric component. Self-cure systems are generally provided to the practitioner as two-part systems, one part comprising the liquid components (e.g., monomer, oligomer, macromonomer and water) and a second part comprising the dry components (e.g., dry cement, initiators, and optional filler).

The curable dental restorative compositions may furthermore optionally comprise a filler component selected from those known in the art of dental restorative materials. Examples of suitable filling materials include but are not limited to, silica, quartz, strontium silicate, strontium borosilicate, lithium silicate, lithium alumina silicate, amorphous silica, ammoniated or deammoniated calcium phosphate, alumina, zirconia, chopped glass fibers, tin oxide, and titania. Particularly suitable fillers are those having a particle size in the range from about 0.1-5.0 μm, mixed with a silicate colloid of 0.001 to about 0.07 microns. Some of the aforementioned inorganic filling materials and methods of preparation thereof are disclosed in U.S. Pat. Nos. 4,544,359 and 4,547,531, pertinent portions of which are incorporated herein by reference. Calcium phosphates preferred in the present invention are, for example, calcium phosphates and tricalcium phosphate.

Certain radiopaque/high refractive index materials, such as apatites, may be used as filler materials. Suitable high refractive index filler materials include, but are not limited to, high refractive index silica glass fillers, calcium silicate based fillers such as apatites, hydroxyapatites or modified hydroxyapatite components. Alternatively, inert, non-toxic radiopaque materials such as bismuth oxide (Bi₂O₃), barium sulfate, and bismuth subcarbonate may be used. Certain fillers, such as glass fillers, may be silanized to improve the bond between filler and resin. The filler is generally present in amounts of about 0 to about 80 percent by weight, preferably about 10 to about 70 percent by weight, and most preferably about 20 to about 60 percent by weight of filler based on the total dental curable composition (i.e., polymerizable component, cement component, curing component, and filler). The amount of filler is readily determined by those of ordinary skill in the art, depending on the intended application and identity of the components.

The dental restorative compositions may further comprise handling agents to aid in dispersion and long-term suspension of the components, particularly the cement. Suitable handling agents may be, for example, viscous materials such as polyethylene glycol (PEG) or polypropylene glycol (PPG), each having a molecular weight of about 400 or greater. Effective quantities of handling agent may be readily determined by one of ordinary skill in the art, depending on the characteristics of the composition and the agent, and may comprise, for example, about 1 to about 50 percent by weight of the total composition, preferably about 10 to about 40 percent by weight of the total composition.

In addition, the dental restorative composition may further comprise other additives, for example anti-oxidants, such as BHT (2,6-di-tert-butyl-4-methylphenol) or hydroquinone methyl ether, in amounts in the range from about 0.1 to about 0.3% by weight of the polymerizable components; ultraviolet stabilizers to prevent discoloration, for example benzophenones such as 2-hydroxy-4-methoxybenzophenone, benzotriazoles such as 2-(2′-hydroxy-5′-methylphenyl)benzotriazole, 2-(2-hydroxy-5-tert-octylphenyl)benzotriazole (available under the trade name UV-54 from American Cyanamid Company) and other derivatives thereof, fluorescent whitening agents such as 2,5-bis(5-tert-butyl-2-benzoxazole) thiophene (available under the trade name UV-OB); trace amounts of FDA and FDC approved dyes, for example carbon black, yellow No. 5, yellow No. 6, and the like; and other additives known in the art such as fluoride, fluoride-releasing agents, polycarboxylic acids useful in the formation of glass ionomer cements such as the homo- and copolymers of acrylic acid and/or itaconic acid, and various medicaments, such as Novocain (procaine hydrochloride), Benzocaine (ethyl aminobenzoate), ascorbic acid, butacaine sulfate, dibucaine hydrochloride, phenobarbital, pentabarbital sodium, butabarbital, diethyl stilbestrol, xylocaine and various known antibiotics.

Use of one or more antibiotics is particularly preferred, as an antibiotic can provide sterilization (which is particularly important in sealants and pulp capping materials) and/or caries prevention (which is particularly important for filling materials). Useful antibiotics include, but are not limited to, one or more of salicylic acid, salicylic acid esters, sulfanilamide, chlorhexidine, erythromycin, bacitracin, hexachloraphene, lincomycin hydrochloride, p-amino salicylic acid, sulfadiazine, procaine penicillin, Aureomycin, streptomycin, tetramycin, chloramphenicol, penicillin, neomycin sulfate, succinoyl-sulfathiazole, cetyl pyridinium chloride, trimethyl benzyl ammonium chloride, triethyl dodecyl ammonium bromide, sulfathiazole, sulfanilamide, and tetracycline. Preferred antibiotics include but are not limited to metronidazole, ciprofloxacin, and minocycline. Other useful antibiotics include amoxicillin, cefroxadine, cefaclor, fosfomycin, or rokitamycin, each of which may be used individually or to replace minocycline.

The antibiotic can be co-polymerized with the dental restorative composition, as disclosed in U.S. Pat. Nos. 5,408,022 and 5,733,949 to Imazato et al., which teaches anti-microbial polymerizable components comprising an ethylenically unsaturated monomer, at least one mono-, di-, or tri-functional ethylenically unsaturated monomer having anti-microbial activity, and a polymerization initiator. Alternatively, the dental restorative materials can be formulated so as to leach the antibiotics to the site to be restored at a controlled rate. Therapeutically effective quantities of antibiotics are readily determined by those of ordinary skill in the art, depending on exemplary factors such as the particular restorative material and use, the strength of the antibiotic, the rate of release from the dental restorative material, cost, and the like. In general, however, therapeutically effective quantities that do not interfere with the advantageous properties of the dental restorative components are between about 0.05 to about 5.0% wt. % of the total composition.

Another preferred embodiment is a method of making a dental restorative comprising preparing a site to be restored in a tooth; and applying the above-describe curable dental restorative composition to the tooth. In one preferred embodiment, the restoration is an endodontically prepared tooth, i.e., a tooth that has been prepared for an endodontic restoration.

The invention is further illustrated by the following non-limiting examples.

### EXAMPLES

Two-part paste-paste formulations A, B, and C, comprising a catalyst part and a base part were prepared in accordance with Table 1 below (all amounts are percent by weight of the total composition):

TABLE 1

| | Formulation | | | | | |
|---|---|---|---|---|---|---|
| | A | | B | | C | |
| Component | Catalyst | Base | Catalyst | Base | Catalyst | Base |
| PEGDMA | 17.1 | — | 19.8 | 24 | 17.1 | — |
| UDMA | 11 | — | 13.2 | — | 11 | — |
| Bis-GMA | — | — | — | 16 | — | — |
| PPG | — | 28.7 | — | — | — | — |
| PEG | — | — | — | — | — | 30 |
| BPO | 0.85 | — | 0.8 | — | 0.85 | — |
| BHT | 0.05 | 0.01 | 0.05 | 0.04 | 0.05 | 0.01 |
| DHEPT | — | 0.29 | — | 0.14 | — | 0.32 |
| BaSO₄ | 40 | — | 65 | — | 40 | — |
| Silica | 1 | 5 | 1.15 | 2.82 | 1 | 5 |
| Filler* | 30 | — | — | 22 | 30 | — |
| Portland | — | 66 | — | 35 | | 64.67 |
| Cement | | | | | | |

*Silane-treated barium glass

Two-part liquid-powder formulations D and E, comprising a catalyst part and a base part were prepared in accordance with Table 2 below (all amounts are percent by weight of the total composition):

TABLE 2

| Formulation | |
|---|---|
| D | E |

| Component | Liquid | Powder | Liquid | Powder |
|---|---|---|---|---|
| Bis-GMA | 15 | | 20 | |
| PEGDMA | 64 | | 79.1 | |
| H₂O | 19.9 | | — | |
| DHEPT | 1 | | 0.8 | |
| BHT | 0.1 | | 0.1 | |
| Portland | | 50 | | 50 |
| Cement | | | | |
| BaSO₄ | | 30 | | 30 |
| Filler* | | 19.4 | | 19.4 |
| BPO | | 0.6 | | 0.6 |

*Silane-treated barium glass

The flexural strength and pH values, and setting times of the various formulations, wherein each part was mixed in a 1:1 ratio, are shown in Table 3 below:

TABLE 3

| | Flexural Strength, | | Setting |
|---|---|---|---|
| Formulation | psi (S.D.) | pH at 72 hours in H₂O | Time, hours |
| A | 1120 (322) | 11.7 | 0.3 |
| B | 8240 (294) | 11.2 | 0.5 |
| C | 1356 (101) | 11.5 | 0.3 |
| D | 2539 (210) | 11.4 | 0.6 |
| E | 5332 (160) | 10.6 | 0.4 |
| ProRoot MTA* | 0** | 11.8 | 10 |

*Control
**The flexural strength of the ProRoot MTA recorded as zero is because there was no load that was able to be recorded when the samples were fractured.

ProRoot MTA is a commercial root canal repair material containing Portland cement, sold by Dentsply Tulsa Dental, Tulsa, Okla., and prepared according to the distributor's instructions. It is believed that there is no polymerizable component or non-water curing component in the system. As may be seen by reference to Table 3, the inventive compositions have far greater flexural strength than the comparative material, and much shorter setting times.

While preferred embodiments have been shown and described, various modifications and substitutions may be made thereto without departing from the spirit and scope of the invention. Accordingly, it is to be understood that the present invention has been described by way of illustration and not limitation.

What is claimed is:

1. A curable, cementitious dental restorative composition, comprising:
a polymerizable, ethylenically unsaturated component;
a hydraulic cement component; and
a non-water curing component.

2. The composition of claim 1, wherein polymerizable component comprises a methacrylate monomer, a methacrylate oligomer, an acrylate monomer, an acrylate oligomer, or a mixture comprising at least one of the foregoing monomers or oligomers.

3. The composition of claim 1, wherein polymerizable component comprises about 5 to about 90 percent by weight of the total weight of the polymerizable component, the cement component, and the non-water curing component.

4. The composition of claim 1, wherein the polymerizable component comprises about 20 to about 70 percent by weight of the total weight of the polymerizable component, the cement component, and the non-water curing component.

5. The composition of claim 1, wherein polymerizable component comprises about 30 to about 60 percent by weight of the total weight of the polymerizable component, the cement component, and the non-water curing component.

6. The composition of claim 1, wherein the cement is a calcium silicate cement.

7. The composition of claim 1, wherein the cement is a Portland cement.

8. The composition of claim 1, wherein the cement comprises, based on the total cement composition, about 50 to about 75 weight percent calcium, calculated as calcium oxide, and about 15 to about 25 weight percent of silicon, calculated on the basis of silicon dioxide.

9. The composition of claim 1, wherein the cement comprises, by weight based on the total composition, and calculated on the basis of the corresponding oxides: 21% SiO₂, 4% Al₂O₃, 5% Fe₂O₃, 65% CaO, 2% MgO, 2.5% SO₃, and 0.5% of alkali oxides.

10. The composition of claim 1, further comprising up to about 50 weight percent of water, based on the weight of the dry cement component.

11. The composition of claim 1, further comprising about 10 to about 40 weight percent of water, based on the weight of the dry cement component.

12. The composition of claim 1, wherein the non-water curing component is a light-curing composition, a self-curing composition, or a combination of the foregoing curing components.

13. The composition of claim 1, further comprising up to about 80 percent by weight of a filler, based on the total weight of the dental restorative composition.

14. The composition of claim 1, further comprising about 10 to about 70 percent by weight of a filler, based on the total weight of the dental restorative composition.

15. The composition of claim 1, further comprising about 20 to about 60 percent by weight of a filler, based on the total weight of the dental restorative composition.

16. The composition of claim 1, in the form of a one-part composition.

17. The composition of claim 1 in the form of a two-part composition, the polymerizable component, and optional water being a first part, and the cement and optional filler being a second part.

18. The composition of claim 1, further comprising a therapeutically effective quantity of an antibiotic.

19. The composition of claim 1, further comprising about 0.05 to 5.0 weight percent of an antibiotic based on the total composition.

20. The composition of claim 13, wherein the antibiotic is one or more of metronidazole, ciprofloxacin, minocycline, amoxicillin, cefroxadine, cefaclor, fosfomycin, or rokitamycin.

21. A curable, cementitious dental restorative composition, comprising:
about 5 to about 90 weight percent of a polymerizable, ethylenically unsaturated component, based on the weight of the polymerizable component, the cement component, and the non-water curing component;
about 10 to about 95 weight percent of a hydraulic, calcium silicate cement component, based on the weight of the polymerizable component, the cement component, and the non-water curing component;
up to about 50 weight percent of water, based on the weight of the dry cement component;
about 0.1 to about 5 weight percent of a non-water curing component, based on the polymerizable component; and up to about 80 percent by weight of filler based on the total weight of the dental restorative composition.

22. The composition of claim 21, wherein the cement is a Portland cement.

23. A method of making a dental restoration, comprising
applying to a tooth the composition comprising a polymerizable, ethylenically unsaturated component, a cement component, and a non-water curing component and
curing the composition.

24. The method of claim 23, wherein polymerizable component comprises a methacrylate monomer, a methacrylate oligomer, an acrylate monomer, an acrylate oligomer, or a mixture comprising at least one of the foregoing monomers or oligomers.

25. The method of claim 23, wherein the cement composition comprises Portland cement.

26. The method of claim 23, wherein the curing composition is a light-curing composition, a self-curing composition, or a combination of the foregoing curing compositions.

27. The method of claim 23, wherein polymerizable component comprises about 5 to about 90 percent by weight of the total weight of the polymerizable component, the cement component, and the non-water curing component.

28. The method of claim 23, wherein the cement is a calcium silicate cement.

29. The method of claim 23, wherein the cement is a Portland cement.

30. The method of claim 23, wherein the cement comprises, based on the total cement composition, about 50 to about 75 weight percent calcium, calculated as calcium oxide, and about 15 to about 25 weight percent of silicon, calculated on the basis of silicon dioxide.

31. The method of claim 23, wherein the cement comprises, by weight based on the total composition, and calculated on the basis of the corresponding oxides: 21% SiO₂, 4% Al₂O₃, 5% Fe₂O₃, 65% CaO, 2% MgO, 2.5% SO₃, and 0.5% of alkali oxides.

32. The method of claim 23, further comprising up to about 50 weight percent of water, based on the weight of the dry cement component.

33. The method of claim 23, wherein the non-water curing component is a light-curing composition, a self-curing composition, or a combination of the foregoing curing components.

34. The method of claim 23, further comprising up to about 80 percent by weight of a filler, based on the total weight of the dental restorative composition.

35. The method of claim 23, wherein the restorative composition further comprises a filler.

36. The method of claim 23, wherein the restorative composition further comprising a therapeutically effective quantity of an antibiotic.

37. The method of claim 23, wherein the antibiotic is one or more of metronidazole, ciprofloxacin, minocycline, amoxicillin, cefroxadine, cefaclor, fosfomycin, or rokitamycin.

38. A dental restoration formed by the method of claim 23.
